(19) United States
(12) Patent Application Publication
(10) Pub. No.: US 2016/0067248 A1
(43) Pub. Date: Mar. 10, 2016

(54) COMBINATIONS

(71) Applicants: GlaxoSmithKline Intellectual Property Development Limited, Brentford (GB); Board of Regents, The University of Texas System, Austin, TX (US)

(72) Inventors: Kurt R. AUGER, Collegeville, PA (US); Justin Bottsford-Miller, Houston, TX (US); Ani Sood, Pearland, TX (US)

(21) Appl. No.: 14/945,818

(22) Filed: Nov. 19, 2015

Related U.S. Application Data
(63) Continuation of application No. 13/979,002, filed on Jul. 10, 2013.
(60) Provisional application No. 61/436,435, filed on Jan. 26, 2011.

(51) Int. Cl. A61K 31/506; A61K 31/337; A61K 31/4439

(52) U.S. Cl. CPC A61K 31/506; A61K 31/4439; A61K 31/337

(57) ABSTRACT

The present invention relates to a method of treating ovarian cancer in a female human and to pharmaceutical combinations useful in such treatment. In particular, the method relates to a ovarian cancer treatment method that includes administering 5-[[4-[(2,3-dimethyl-2H-indazol-6-yl)methylamino]-2-pyrimidinyl]amino]-2-methylbenzenesulfonamide, or a pharmaceutically acceptable salt thereof, and 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof, and optionally 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate}, to a human in need thereof.

This application is a Continuation of U.S. application Ser. No. 13/979,002 filed Jul. 10, 2013, which is a 371 of International Application No. PCT/US2012/022638 filed Jan. 26, 2012, which claims priority to U.S. Provisional Application No. 61/436,435 filed Jan. 26, 2011 which are incorporated herein in their entirety.

### FIELD OF THE INVENTION

The present invention relates to a method of treating cancer in a mammal and to combinations useful in such treatment. In particular, the method relates to a novel combination comprising a VEGFR inhibitor and a focal adhesion kinase inhibitor and/or an microtubule inhibitor, pharmaceutical compositions comprising the same, and methods of using such combinations in the treatment of cancer.

### BACKGROUND OF THE INVENTION

Generally, cancer results from the deregulation of the normal processes that control cell division, differentiation and apoptotic cell death. Apoptosis (programmed cell death) plays essential roles in embryonic development and pathogenesis of various diseases, such as degenerative neuronal diseases, cardiovascular diseases and cancer. One of the most commonly studied pathways, which involves kinase regulation of apoptosis, is cellular signaling from growth factor receptors at the cell surface to the nucleus (Crews and Erikson, Cell, 74:215-17, 1993).

The process of angiogenesis is the development of new blood vessels from the pre-existing vasculature. Angiogenesis is defined herein as involving: (i) activation of endothelial cells; (ii) increased vascular permeability; (iii) subsequent dissolution of the basement membrane and extravasation of plasma components leading to formation of a provisional fibrin gel extracellular matrix; (iv) proliferation and mobilization of endothelial cells; (v) reorganization of mobilized endothelial cells to form functional capillaries; (vi) capillary loop formation; and (vi) deposition of basement membrane and recruitment of perivascular cells to newly formed vessels. Normal angiogenesis is active during tissue growth from embryonic development through maturity and then enters a period of relative quiescence during adulthood. Normal angiogenesis is also activated during wound healing, and at certain stages of the female reproductive cycle. Inappropriate or pathological angiogenesis has been associated with several disease states including various retinopathies, ischemic disease, atherosclerosis, chronic inflammatory disorders, and cancer. The role of angiogenesis in disease states is discussed, for instance, in Fan et al., Trends in Pharmacol Sci. 16:54-66; Shawver et al., DDT Vol. 2, No. 2 Feb. 1997; Folkmann, 1995, Nature Medicine 1:27-31.

In cancer the growth of solid tumors has been shown to be dependent on angiogenesis. The progression of leukemias as well as the accumulation of fluid associated with malignant ascites and pleural effusions also involve pro-angiogenic factors. (See Folkmann, J., J. Nat'l. Cancer Inst, 1990, 82, 4-6).

Central to the process of angiogenesis are vascular endothelial growth factor (VEGF) and its receptors, termed vascular endothelial growth factor receptor(s) (VEGFRs). The roles VEGF and VEGFRs play in the vascularization of solid tumors, progression of hematopoietic cancers and modulation of vascular permeability have drawn great interest in the scientific community. VEGF is a polypeptide, which has been linked to inappropriate or pathological angiogenesis (Pinedo, H. M. et al. The Oncologist, Vol. 5, No. 90001, 1-2, Apr. 2000). VEGFR(s) are protein tyrosine kinases (PTKs) that catalyze the phosphorylation of specific tyrosine residues in proteins that are involved in the regulation of cell growth, differentiation, and survival. (A. F. Wilks, Progress in Growth Factor Research, 1990, 2, 97-111; S. A. Courtneidge, Dev. Supp. 1, 1993, 57-64; J. A. Cooper, Semin. Cell Biol., 1994, 5(6), 377-387; R. F. Paulson, Semin. Immunol. 1995, 7(4), 267-277; A. C. Chan, Curr. Opin. Immunol. 1996, 8(3), 394-401).

Three PTK receptors for VEGF have been identified: VEGFRI (Flt-I); VEGFR2 (Flk-I and KDR) and VEGFR3 (Flt-4). These receptors are involved in angiogenesis and participate in signal transduction. (Mustonen, T. et al. J. Cell. Biol. 1995: 129:895-898; Ferrara and Davis-Smyth, Endocrine Reviews, 18(1):4-25, 1997; McMahon, G., The Oncologist, Vol. 5, No 90001, 3-10, Apr. 2000).

Of particular interest is VEGFR2, which is a transmembrane receptor PTK expressed primarily in endothelial cells. Activation of VEGFR-2 by VEGF is a critical step in the signal transduction pathway that initiates tumor angiogenesis. VEGF expression may be constitutive to tumor cells and can also be upregulated in response to certain stimuli. One such stimulus is hypoxia, where VEGF expression is upregulated in both tumor and associated host tissues. The VEGF ligand activates VEGFR2 by binding to its extracellular VEGF binding site. This leads to receptor dimerization of VEGFRs and autophosphorylation of tyrosine residues at the intracellular kinase domain of VEGFR2. The kinase domain operates to transfer a phosphate from ATP to the tyrosine residues, thus providing binding sites for signaling proteins downstream of VEGFR-2 leading ultimately to angiogenesis. (Ferrara and Davis-Smyth, Endocrine Reviews, 18(1):4-25, 1997; McMahon, G. The Oncologist, Vol. 5, No. 90001, 3-10, Apr. 2000.)

Consequently, antagonism of the VEGFR2 kinase domain would block phosphorylation of tyrosine residues and serve to disrupt initiation of angiogenesis. Specifically, inhibition at the ATP binding site of the VEGFR2 kinase domain would prevent binding of ATP and prevent phosphorylation of tyrosine residues. Such disruption of the proangiogenesis signal transduction pathway associated with VEGFR2 should therefore inhibit tumor angiogenesis and thereby provide a potent treatment for cancer or other disorders associated with inappropriate angiogenesis. Votrient (pazopanib hydrochloride) is a multi-tyrosine kinase inhibitor of vascular endothelial growth factor receptor (VEGFR)-1, VEGFR-2, VEGFR-3, platelet-derived growth factor receptor (PDGFR)-α and -β, fibroblast growth factor receptor (FGFR)-1 and -3, cytokine receptor (Kit), interleukin-2 receptor inducible T-cell kinase (Itk), leukocyte-specific protein tyrosine kinase (Lck), and transmembrane glycoprotein receptor tyrosine kinase (c-Fms) and is approved in the US for the treatment of patients with advanced renal cell carcinoma. The chemical name of pazopanib hydrochloride is 5-[[4-[(2,3-dimethyl-2H-indazol-6-yl)methylamino]-2-pyrimidinyl]amino]-2-methylbenzenesulfonamide monohydrochloride.

Tyrosine kinases play an important role in the regulation of many cell processes including cell proliferation, cell survival, and cell migration. It is known that certain tyrosine kinases become activated by mutation or are abnormally expressed in many human cancers. For example, the epidermal growth factor receptor (EGFR) is found mutated and/or overexpressed in breast, lung, brain, squamous cell, gastric, and other human cancers. Selective inhibitors of the tyrosine kinase activity of EGFR have been shown to be of clinical value in treatment of cancers with mutated and/or overexpressed EGFR. Thus, selective inhibitors of particular tyrosine kinases are useful in the treatment of proliferative diseases such as cancer.

FAK (encoded by the gene PTK2) is a non-receptor tyrosine kinase that integrates signals from integrins and growth factor receptors. FAK has been reported to playa role in the regulation of cell survival, growth, adhesion, migration, and invasion (McLean et al 2005, Nat Rev Cancer 20 5:505-515). Furthermore, FAK is regulated and activated by phosphorylation on multiple tyrosine residues. Overexpression of FAK mRNA and/or protein has been documented in many solid human tumors, including but not limited to, cancers of the breast, colon, thyroid, lung, ovary, and prostate; but also including cancers of hematological origin, including but not limited to leukemia such as acute myeloid leukemia (AML). (Owens et al. 1995, Cancer Research 55: 2752-2755; Agochiya et al. 1999, Oncogene 18: 5646-5653; Gabarro-Niecko et al. 2003, Cancer Metastasis Rev. 22:359-374; Recher et al. 2004, Cancer Research 64:3191-3197; Zhao and Guan, 28:35-49, 2009, Cancer Metastasis Rev.). More significantly, there is evidence that phosphorylated FAK is increased in malignant compared to normal tissues (Grisaru-Granovsky et al. 2005, Int. J. Cancer 113: 372-378) and could represent a prognostic marker of metastasis. FAK activity is clearly implicated in advanced and metastatic human cancer (Zhao and Guan, 28:35-49, 2009, Cancer Metastasis Rev.).

Elimination of FAK by RNAi or expression of a FAK dominant negative has been shown to induce loss of adhesion and cell death in human breast and melanoma cell lines, and to augment docetaxel-mediated apoptosis in ovarian cancer cells (Beviglia et a12003, Biochem J. 373:201-210, Smith et al. 2005, Melanoma Res. 15:357-362, Halder et a12005, Clin. Cancer Res. 11:88298836). However, inhibition of FAK in normal human fibroblasts or immortalized mammary cells (MCFIOA) was found not to cause loss of attachment or apoptosis (Xu et al. 1996 Cell Growth and Diff 7:413-418). Inhibition of FAK by dominant negative expression has also been shown to reduce tumor growth and eliminate lung metastasis of mammary adenocarcinoma cells in a syngeneic rat model (van Nimwegen et a12005, Cancer Res. 65:4698-4706). Similarly, inhibition of FAK by shRNA inhibited lung metastasis and reduced lethality by 40% in a syngeneic mouse model (Mitra et al 2006, Oncogene 25: 4429-4440). In this study, transient re-expression of wildtype, but not kinase-dead FAK, reversed the shRNA phenotypes. Inhibition of FAK by dominant negative expression in mouse 4TI carcinoma cells reduced tumor growth and angiogenesis in mice (Mitra et a12006, Oncogene 25:5969-5984). Furthermore, loss ofFAK catalytic activity (reconstitution of FAK-I-cells with kinase-dead FAK) reduced growth ofv-Src tumors in mice and decreased angiogenesis.

Previous researchers have noted that docetaxel (TAXOTERE®) and its derivatives (such as TAXOL®, paclitaxel) are useful in the treatment of the malignant neoplasms, such as solid tumors and other malignancies. European Patent EP 0 253 738 and International Patent Application WO 92/09589 describe a method of preparation of docetaxel. Generally, the doses, which vary depending on the patient, comprise between 60 and 400 mg/m² of docetaxel. Commonly, docetaxel is administered via intravenous route at doses of 60 to 100 mg/m² over 1 hour every 3 weeks (Textbook of Medical Oncology, Franco Cavelli et al., Martin Dunitz Ltd., p. 4623 (1997)).

Many clinical studies have confirmed the efficacy of docetaxel in treating many types of cancer, particularly breast cancer. Docetaxel's effects are shown in both first and second line therapies. The mechanism of docetaxel's action is thought to be via enhancement of microtubule assembly and inhibition of the depolymerization of tubulin at the cellular level.

It would be useful to provide a novel therapy which provides more effective and/or enhanced treatment of an individual suffering the effects of cancer.

### SUMMARY OF THE INVENTION

One embodiment of this invention provides a combination that includes:
(i) a compound of Structure (I):

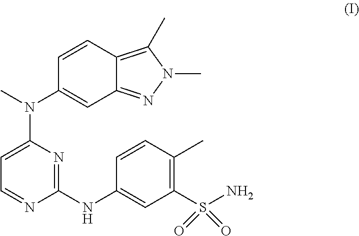 [SMILES: Cc1ccc(Nc2nccc(N(C)c3ccc4c(C)n(C)nc4c3)n2)cc1S(N)(=O)=O]

or a pharmaceutically acceptable salt thereof; and
(ii) a compound of Structure (II):

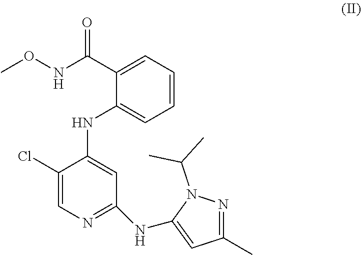

or a pharmaceutically acceptable salt thereof.

Another embodiment of this invention provides a combination that includes:
(i) a compound of Structure (I):

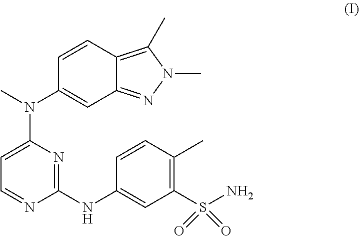 [SMILES: Cc1ccc(Nc2nccc(N(C)c3ccc4c(C)n(C)nc4c3)n2)cc1S(N)(=O)=O]

or a pharmaceutically acceptable salt thereof;
(ii) a compound of Structure (II):

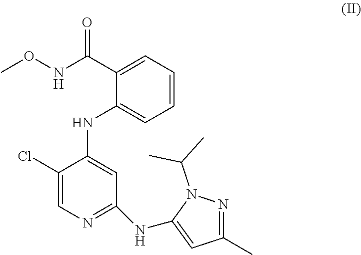

or a pharmaceutically acceptable salt thereof; and
(iii) a compound of Structure (Ill):

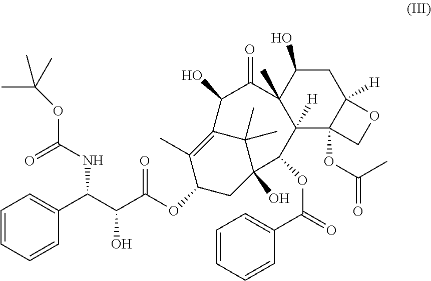

One embodiment of this invention provides a method of treating ovarian cancer in a female human in need thereof which comprises the in vivo administration of a therapeutically effective amount of a combination of 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof, to such human.

Another embodiment of this invention provides a method of treating ovarian cancer in a female human in need thereof which comprises the in vivo administration of a therapeutically effective amount of a combination of 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof, and 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate} to such human.

One embodiment of this invention provides a method of treating ovarian cancer in a female human in need thereof which comprises the in vivo administration of a therapeutically effective amount of a combination of 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof, to such human, wherein the combination is administered within a specified period, and wherein the combination is administered for a duration of time.

Another embodiment of this invention provides a method of treating ovarian cancer in a female human in need thereof which comprises the in vivo administration of a therapeutically effective amount of a combination of 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof, and 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate} to such human, wherein the combination is administered within a specified period, and wherein the combination is administered for a duration of time.

One embodiment of this invention provides a method of treating ovarian cancer in a female human in need thereof which comprises the in vivo administration of a therapeutically effective amount of a combination of 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof, to such human, wherein the compounds of the combination are administered sequentially.

Another embodiment of this invention provides a method of treating ovarian cancer in a female human in need thereof which comprises the in vivo administration of a therapeutically effective amount of a combination of 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof, and 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate} to such human, wherein the compounds of the combination are administered sequentially.

### BRIEF DESCRIPTION OF THE DRAWINGS

FIG. 1 illustrates the tumor mass for animal groups treated with control, pazopanib monotherapy, FAK inhibitor monotherapy, docetaxel monotherapy, combination of pazopanib and FAK inhibitor, combination of pazopanib and docetaxel, combination of FAK inhibitor and docetaxel, and the triple combination of pazopanib, FAK inhibitor and docetaxel;

FIG. 2 illustrates mean ascites volume for animal groups treated with control, pazopanib monotherapy, FAK inhibitor monotherapy, docetaxel monotherapy, combination of pazopanib and FAK inhibitor, combination of pazopanib and docetaxel, combination of FAK inhibitor and docetaxel, and the triple combination of pazopanib, FAK inhibitor and docetaxel; and

FIG. 3 illustrates mean number of tumor nodules for animal groups treated with control, pazopanib monotherapy, FAK inhibitor monotherapy, docetaxel monotherapy, combination of pazopanib and FAK inhibitor, combination of pazopanib and docetaxel, combination of FAK inhibitor and docetaxel, and the triple combination of pazopanib, FAK inhibitor and docetaxel.

### DETAILED DESCRIPTION OF THE INVENTION

The present invention relates to combinations that exhibit antitumor activity. In some embodiments, the method relates to methods of treating ovarian cancer by the co-administration of 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, (hereinafter Compound A, or a pharmaceutically acceptable salt, suitably the monohydrochloride salt, thereof), which compound is represented by Structure I:

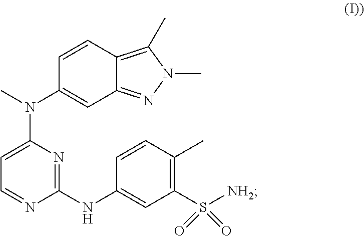 [SMILES: Cc1ccc(Nc2nccc(N(C)c3ccc4c(C)n(C)nc4c3)n2)cc1S(N)(=O)=O]

and 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof (hereinafter Compound B or a pharmaceutically acceptable salt thereof), which compound is represented by Structure II:

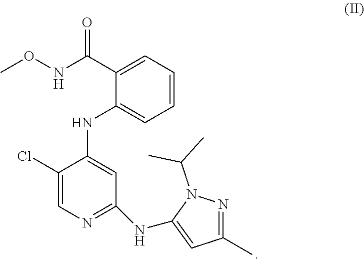

Compound A is disclosed and claimed, along with pharmaceutically acceptable salts thereof, as being useful as an inhibitor of VEGFR activity, particularly in treatment of cancer, in International Application No. PCT/US01/49367, having an International filing date of Dec. 19, 2001, International Publication Number WO02/059110 and an International Publication date of Aug. 1, 2002, the entire disclosure of which is hereby incorporated by reference, Compound A is the compound of Example 69. Compound A can be prepared as described in International Application No. PCT/US01/49367.

Suitably, Compound A is in the form of a monohydrochloride salt. This salt form can be prepared by one of skilled in the artskilled in the art from the description in International Application No. PCT/US01/49367, having an International filing date of Dec. 19, 2001.

Compound A is sold commercially as the monohydrochloride salt. Compound A is known by the generic name pazopanib and the trade name Votrient®.

Compound B is disclosed and claimed, along with pharmaceutically acceptable salts thereof, as being useful as an inhibitor of focal adhesion kinase, particularly in treatment of cancer, in International Publication No. WO2010/062578 having a filing date of Oct. 27, 2009, the entire disclosure of which is hereby incorporated by reference, Compound B is the compound of Example 41. Compound B can be prepared as described in this patent application.

In other embodiments, the method relates to methods of treating ovarian cancer by the co-administration of Compound A, or a pharmaceutically acceptable salt, suitably the monohydrochloride salt, thereof, Compound B, or a pharmaceutically acceptable salt thereof, and 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate} (hereinafter Compound C), which compound is represented by Structure III:

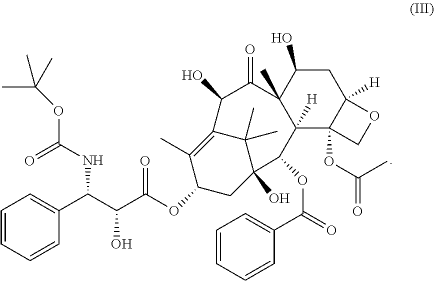

Compound C is disclosed as being a microtubule inhibitor, particularly in the treatment of cancer, and claimed U.S. Pat. No. 4,814,470, filed Jul. 14, 1987, the entire disclosure of which is hereby incorporated by reference. Compound C can be prepared as described in U.S. Pat. No. 4,814,470.

Compound C is known by the generic name docetaxel and the trade name Taxotere®.

The administration of a therapeutically effective amount of the combinations of the invention are advantageous over the individual component compounds in that the combinations will provide one or more of the following improved properties when compared to the individual administration of a therapeutically effective amount of a component compound: i) a greater anticancer effect than the most active single agent, ii) synergistic or highly synergistic anticancer activity, iii) a dosing protocol that provides enhanced anticancer activity with reduced side effect profile, iv) a reduction in the toxic effect profile, v) an increase in the therapeutic window, and/or vi) an increase in the bioavailability of one or both of the component compounds.

The compounds of the invention may contain one or more chiral atoms, or may otherwise be capable of existing as two enantiomers. Accordingly, the compounds of this invention include mixtures of enantiomers as well as purified enantiomers or enantiomerically enriched mixtures. Also, it is understood that all tautomers and mixtures of tautomers are included within the scope of Compound A, and pharmaceutically acceptable salts thereof, Compound B, and pharmaceutically acceptable salts thereof, and Compound C.

The compounds of the invention may form a solvate which is understood to be a complex of variable stoichiometry formed by a solute (in this invention, Compound A or a salt thereof, Compound B or a salt thereof, and/or Compound C) and a solvent. Such solvents for the purpose of the invention may not interfere with the biological activity of the solute. Examples of suitable solvents include, but are not limited to, water, methanol, ethanol and acetic acid. Suitably the solvent used is a pharmaceutically acceptable solvent. Suitably the solvent used is water.

The pharmaceutically acceptable salts of the compounds of the invention are readily prepared by those of skilled in the art.

Also, contemplated herein is a method of treating ovarian cancer using a combination of the invention where Compound A, or a pharmaceutically acceptable salt thereof, and/or Compound B or a pharmaceutically acceptable salt thereof are administered as pro-drugs. Pharmaceutically acceptable pro-drugs of the compounds of the invention are readily prepared by those of skilled in the art.

Additionally, contemplated herein is a method of treating ovarian cancer using a combination of the invention where Compound A, or a pharmaceutically acceptable salt thereof, Compound B or a pharmaceutically acceptable salt thereof, and/or Compound C are administered as pro-drugs. Pharmaceutically acceptable pro-drugs of the compounds of the invention are readily prepared by those of skilled in the art.

When referring to a dosing protocol, the term “day”, “per day” and the like, refer to a time within one calendar day which begins at midnight and ends at the following midnight.

By the term “treating” and derivatives thereof as used herein, is meant therapeutic therapy. In reference to a particular condition, treating means: (1) to ameliorate the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition.

Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing ovarian cancer, such as when a subject has a strong family history of ovarian cancer or when a subject has been exposed to a carcinogen.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

By the term “combination” and derivatives thereof, as used herein is meant either, simultaneous administration or any manner of separate sequential administration of a therapeutically effective amount of Compound A, or a pharmaceutically acceptable salt thereof, and Compound B or a pharmaceutically acceptable salt thereof, and, in some embodiments, additionally Compound C. Preferably, if the administration is not simultaneous, the compounds are administered in a close time proximity to each other. Furthermore, it does not matter if the compounds are administered in the same dosage form, e.g. one compound may be administered topically and the other compound may be administered orally. Suitably, compounds A and B are administered orally, and Compound C is administered via intravenous or intraperitoneal route.

By the term “combination kit” as used herein is meant the pharmaceutical composition or compositions that are used to administer Compound A, or a pharmaceutically acceptable salt thereof, and Compound B, or a pharmaceutically acceptable salt thereof, according to the invention, and in some embodiments, additionally Compound C. When Compound A, or a pharmaceutically acceptable salt thereof, and Compound B, or a pharmaceutically acceptable salt thereof, are administered simultaneously, the combination kit can contain Compound A, or a pharmaceutically acceptable salt thereof, and Compound B, or a pharmaceutically acceptable salt thereof, in a single pharmaceutical composition, such as a tablet, or in separate pharmaceutical compositions, and, in some embodiments, additionally contain Compound C in a form suitable for intravenous or intraperitoneal administration, such as a concentrated form capable of dilution and administration. When Compound A, or a pharmaceutically acceptable salt thereof, and Compound B, or a pharmaceutically acceptable salt thereof, are not administered simultaneously, the combination kit will contain Compound A, or a pharmaceutically acceptable salt thereof, and Compound B, or a pharmaceutically acceptable salt thereof, in separate pharmaceutical compositions, and, in some embodiments, additionally contain Compound C in a form suitable for intravenous or intraperitoneal administration, such as a concentrated form capable of dilution and administration. The combination kit can comprise Compound A, or a pharmaceutically acceptable salt thereof, and Compound B, or a pharmaceutically acceptable salt thereof, in separate pharmaceutical compositions in a single package or in separate pharmaceutical compositions in separate packages, and, in some embodiments, further comprise Compound C in a form suitable for intravenous or intraperitoneal administration, such as a concentrated form capable of dilution and administration.

In one aspect there is provided a combination kit comprising the components: Compound A, or a pharmaceutically acceptable salt thereof, in association with a pharmaceutically acceptable carrier; and Compound B, or a pharmaceutically acceptable salt thereof, in association with a pharmaceutically acceptable carrier.

In one embodiment of the invention the combination kit comprises the following components: Compound A, or a pharmaceutically acceptable salt thereof, in association with a pharmaceutically acceptable carrier; and Compound B, or a pharmaceutically acceptable salt thereof, in association with a pharmaceutically acceptable carrier, wherein the components are provided in a form which is suitable for sequential, separate and/or simultaneous administration.

In one embodiment the combination kit comprises: a first container comprising Compound A, or a pharmaceutically acceptable salt thereof, in association with a pharmaceutically acceptable carrier; and a second container comprising Compound B, or a pharmaceutically acceptable salt thereof, in association with a pharmaceutically acceptable carrier, and a container means for containing said first and second containers.

In another aspect there is provided a combination kit comprising the components: Compound A, or a pharmaceutically acceptable salt thereof, in association with a pharmaceutically acceptable carrier; Compound B, or a pharmaceutically acceptable salt thereof, in association with a pharmaceutically acceptable carrier, and Compound C in a form suitable for intravenous or intraperitoneal administration, such as a concentrated form capable of dilution and administration.

In one embodiment of the invention the combination kit comprises the following components: Compound A, or a pharmaceutically acceptable salt thereof, in association with a pharmaceutically acceptable carrier; Compound B, or a pharmaceutically acceptable salt thereof, in association with a pharmaceutically acceptable carrier, and Compound C in a form suitable for intravenous or intraperitoneal administration, such as a concentrated form capable of dilution and administration. wherein the components are provided in a form which is suitable for sequential, separate and/or simultaneous administration.

In one embodiment the combination kit comprises: a first container containing Compound A, or a pharmaceutically acceptable salt thereof, in association with a pharmaceutically acceptable carrier; and a second container containing Compound B, or a pharmaceutically acceptable salt thereof, in association with a pharmaceutically acceptable carrier, and a third container containing Compound C in a form suitable for intravenous or intraperitoneal administration, such as a concentrated form capable of dilution and administration, and a container means for containing said first, second and third containers.

The “combination kit” can also be provided by instruction, such as dosage and administration instructions. Such dosage and administration instructions can be of the kind that is provided to a doctor, for example by a drug product label, or they can be of the kind that is provided by a doctor, such as instructions to a patient.

As used herein the term “Compound A²” means—Compound A, or a pharmaceutically acceptable salt thereof—.

As used herein the term “Compound B²” means—Compound B, or a pharmaceutically acceptable salt thereof—.

In some embodiments according to the present invention, the combinations of this invention are administered within a “specified period”.

When the combination of compound A² and Compound B², without Compound C, is administered, the term “specified period” and derivatives thereof, as used herein means the interval of time between the administration of one of Compound A² and Compound B² and the other of Compound A² and Compound B². Unless otherwise defined, the specified period can include simultaneous administration. When Compound A² and Compound B² are administered once a day the specified period refers to timing of the administration of Compound A² and Compound B² during a single day. When one or both compounds of the invention are administered more than once a day, the specified period is calculated based on the first administration of each compound on a specific day. All administrations of a compound of the invention that are subsequent to the first during a specific day are not considered when calculating the specific period.

The specified period can be various time periods. For example, Compound A² and Compound B² can be administered within about 24, 23, 22, 21, 20, 19, 18, 17, 16, 15, 14, 13, 12, 11, 10, 9, 8, 7, 6, 5, 4, 3, 2 or 1 hours of each other, in which case the specified period will be about 24, 23, 22, 21, 20, 19, 18, 17, 16, 15, 14, 13, 12, 11, 10, 9, 8, 7, 6, 5, 4, 3, 2 or 1 hours, respectively. As used herein, the administration of Compound A² and Compound B² in less than about 45 minutes apart is considered simultaneous administration.

When the combination of compound A², Compound B² and Compound C is administered, the term “specified period” and derivatives thereof, as used herein means the interval of time between the administration of one of Compound A², Compound B², and Compound C, and the first administration of the last of Compound A², Compound B², and Compound C to be administered. Unless otherwise defined, the specified period can include simultaneous administration. When compounds A², B² and C are administered once a day the specified period refers to timing of the administration of Compound A², Compound B² and Compound C during a single day. When one or more of compounds A², B² and C are administered more than once a day, the specified period is calculated based on the first administration of each compound on a specific day. All administrations of a compound of the invention that are subsequent to the first during a specific day are not considered when calculating the specific period.

The specified period can be various time periods. For example, Compound A², Compound B² and Compound C can be administered within about 24, 23, 22, 21, 20, 19, 18, 17, 16, 15, 14, 13, 12, 11, 10, 9, 8, 7, 6, 5, 4, 3, 2 or 1 hours of each other, in which case the specified period will be about 24, 23, 22, 21, 20, 19, 18, 17, 16, 15, 14, 13, 12, 11, 10, 9, 8, 7, 6, 5, 4, 3, 2 or 1 hours, respectively. As used herein, the administration of Compound A², Compound B², and Compound C in less than about 45 minutes apart is considered simultaneous administration.

Suitably, when the combination of the invention is administered for a “specified period,” the compounds will be co-administered for a “duration of time.”

When the combination of compound A² and Compound B², without Compound C, is administered, the term “duration of time” and derivatives thereof, as used herein means that Compound A² and Compound B² are administered within a “specified period” for an indicated number of consecutive days, optionally followed by a number of consecutive days where only one of the component compounds is administered. Unless otherwise defined, the “duration of time” in all dosing protocols described herein, does not have to commence with the start of treatment and terminate with the end of treatment, it is only required that the number of consecutive days in which both compounds are administered and the optional number of consecutive days in which only one of the component compounds is administered, or the indicated dosing protocol, occur at some point during the course of treatment.

The duration of time can be various time periods. For example, Compound A² and Compound B² can both be administered within a specified period for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 consecutive days during the course of treatment, in which case the duration of time will be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, respectively. When, during the course of treatment, both compounds are administered within a specified period for over 30 consecutive days, the treatment is considered chronic treatment and will continue until an altering event, such as a reassessment in ovarian cancer status or a change in the condition of the patient, warrants a modification to the protocol.

Various treatment protocols are contemplated in embodiments of the present invention. For example, Compound A² and B² can be co-administered within a specified period for at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days, followed by the administration of Compound A² alone for at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days, in which case the duration of time will be at least the number of consecutive days that Compound A² and Compound B² are both administered plus the number of consecutive days of administration of Compound A² alone (e.g., if Compound A² and Compound B² are both administered for 6 consecutive days followed by administration of Compound A² alone for 8 consecutive days, the duration of time will be at least 14 consecutive days).

In other embodiments, Compound A² and Compound B² are both administered within a specified period for a number of consecutive days during a certain time period, and compound A² is administered during the other days of the certain time period. In some embodiments, the certain time period is n=2, 3, 4, 5, 6, 7, 8, 9, 10, 11, 12, 13, 14, 15, 16, 17, 18, 19, 20, 21, 22, 23, 24, 25, 26, 27, 28, 29 or 30 days, the days of consecutive administration of Compound A² and Compound B² within a specified time period is m=1, 2, 3, 4, 5, 6, 7, 8, 9, 10, 11, 12, 13, 14, 15, 16, 17, 18, 19, 20, 21, 22, 23, 24, 25, 26, 27, 28 or 29, and the days of administration of Compound A² is n−m, where n−m is at least 1. For example, Compound A² and Compound B² can be administered within a specified time period for 1, 2, 3, 4, 5, 6, 7, 8, 9, 10, 11, 12 or 13 consecutive days over a certain time period of 14 days, during which Compound A² is administered for the other 13, 12, 11, 10, 9, 8, 7, 6, 5, 4, 3, 2 or 1 days, respectively. In this example, n=14, m=1, 2, 3, 4, 5, 6, 7, 8, 9, 10, 11, 12 or 13, and n−m=13, 12, 11, 10, 9, 8, 7, 6, 5, 4, 3, 2 or 1, respectively. The consecutive days during which Compound A² and Compound B² are both administered within a specified time period can occur any time during the certain time period. Accordingly, in the foregoing example, Compound A² could be administered alone for 4 consecutive days follow by administration of both Compound A² and Compound B² for 5 consecutive days, followed by administering Compound A² alone for 5 consecutive days to complete the 14 day certain time period.

While treatment protocols have been described with respect to administration of both Compound A² and Compound B² within a specified period in conjunction with administration of Compound A² alone, embodiments of the present invention also include similar treatment protocols in which Compound A² and Compound B² are both administered within a specified period in conjunction with administration of Compound B² alone.

Other embodiments of the present invention include administration of both Compound A² and Compound B² within a specified period in conjunction with administration of Compound A² alone and administration of Compound B² alone. For example, in some embodiments Compound A² and Compound B² are both administered within a specified period for a number of consecutive days during a certain time period, Compound A² is administered alone during a number of days during the certain time period, and Compound B² is administered alone during the other days during the certain time period. In some embodiments, the certain time period is n=3, 4, 5, 6, 7, 8, 9, 10, 11, 12, 13, 14, 15, 16, 17, 18, 19, 20, 21, 22, 23, 24, 25, 26, 27, 28, 29 or 30 days, the days of consecutive administration of Compound A² and Compound B² within a specified time period is m=1, 2, 3, 4, 5, 6, 7, 8, 9, 10, 11, 12, 13, 14, 15, 16, 17, 18, 19, 20, 21, 22, 23, 24, 25, 26, 27 or 28, the days of administration of Compound A² during the certain time period is p=1, 2, 3, 4, 5, 6, 7, 8, 9, 10, 11, 12, 13, 14, 15, 16, 17, 18, 19, 20, 21, 22, 23, 24, 25, 26, 27 or 28, and the days of administration of Compound B² is n−m−p, where n−m−p is at least 1. For example, Compound A² and Compound B² can both be administered within a specified time period for 1, 2, 3, 4, 5, 6, 7, 8, 9, 10, 11 or 12 consecutive days over a certain time period of 14 days, during which Compound A² is administered for 1, 2, 3, 4, 5, 6, 7, 8, 9, 10, 11 or 12 days, and Compound B² is administered for 1, 2, 3, 4, 5, 6, 7, 8, 9, 10, 11 or 12 days. In this example, n=14, m=1, 2, 3, 4, 5, 6, 7, 8, 9, 10, 11 or 12, p=1, 2, 3, 4, 5, 6, 7, 8, 9, 10, 11 or 12, and n−m−p=1, 2, 3, 4, 5, 6, 7, 8, 9, 10, 11 or 12. The consecutive days during which Compound A² and Compound B² are both administered within a specified time period can occur any time during the certain time period. Accordingly, in the foregoing example, Compound A² could be administered alone for 4 consecutive days follow by administration of both Compound A² and Compound B² for 5 consecutive days, followed by administering Compound B² alone for 5 consecutive days to complete the 14 day certain time period. Administration of Compound A² alone and administration of Compound B² alone do not have to occur on consecutive days. Accordingly, in the foregoing example, Compound A² could be administered for 2 consecutive days, followed by administration of Compound B² for 1 day followed by administration of both Compound A² and Compound B² for 5 consecutive days, followed by administration of Compound A² for 1 day, followed by administration of Compound B² for 5 consecutive days.

When the combination of compound A², Compound B², and Compound C is administered, the term “duration of time” and derivatives thereof, as used herein means that Compound A², Compound B² and Compound C are administered within a “specified period” for an indicated number of consecutive days, optionally followed by a number of consecutive days where only one or two of the component compounds is administered. Unless otherwise defined, the “duration of time” in all dosing protocols described herein, does not have to commence with the start of treatment and terminate with the end of treatment, it is only required that the number of consecutive days in which Compound A², Compound B² and Compound C are administered and the optional number of consecutive days in which only one or two of the component compounds is administered, or the indicated dosing protocol, occur at some point during the course of treatment.

The duration of time can be various time periods. For example, Compound A², Compound B² and Compound C can be administered within a specified period for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 consecutive days during the course of treatment, in which case the duration of time will be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, respectively. When, during the course of treatment, the three compounds are administered within a specified period for over 30 consecutive days, the treatment is considered chronic treatment and will continue until an altering event, such as a reassessment in ovarian cancer status or a change in the condition of the patient, warrants a modification to the protocol.

Various treatment protocols are contemplated in embodiments of the present invention. For example, Compounds A², B² and C can be co-administered within a specified period for at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days, followed by the administration of one or two of Compounds A², B² and C for at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days, in which case the duration of time will be at least the number of consecutive days that Compounds A², B² and C are administered plus the number of consecutive days of administration of one or two of Compounds A², B² and C (e.g., if Compounds A², B² and C are administered for 6 consecutive days followed by administration of Compound A² alone for 8 consecutive days, the duration of time will be at least 14 consecutive days, and if Compounds A², B² and C are administered for 7 consecutive days followed by administration of Compound A² and C for 10 consecutive days, the duration of time will be at least 17 consecutive days).

In other embodiments, Compounds A², B² and C are administered within a specified period for a number of consecutive days during a certain time period, and one or two of Compounds A², B² and C is administered during the other days of the certain time period. In some embodiments, the certain time period is n=2, 3, 4, 5, 6, 7, 8, 9, 10, 11, 12, 13, 14, 15, 16, 17, 18, 19, 20, 21, 22, 23, 24, 25, 26, 27, 28, 29 or 30 days, the days of consecutive administration of Compounds A², B² and C within a specified time period is m=1, 2, 3, 4, 5, 6, 7, 8, 9, 10, 11, 12, 13, 14, 15, 16, 17, 18, 19, 20, 21, 22, 23, 24, 25, 26, 27, 28 or 29, and the days of administration of one or two of Compounds A², B² and C is n−m, where n−m is at least 1. For example, Compounds A², B² and C can be administered within a specified time period for 1, 2, 3, 4, 5, 6, 7, 8, 9, 10, 11, 12 or 13 consecutive days over a certain time period of 14 days, during which Compound B² is administered for the other 13, 12, 11, 10, 9, 8, 7, 6, 5, 4, 3, 2 or 1 days, respectively. In this example, n=14, m=1, 2, 3, 4, 5, 6, 7, 8, 9, 10, 11, 12 or 13, and n−m=13, 12, 11, 10, 9, 8, 7, 6, 5, 4, 3, 2 or 1, respectively. The consecutive days during which Compounds A², B² and C are administered within a specified time period can occur any time during the certain time period. Accordingly, in the foregoing example, Compound B² could be administered alone for 4 consecutive days follow by administration of Compounds A², B² and C for 5 consecutive days, followed by administration of Compound B² alone for 5 consecutive days to complete the 14 day certain time period.

In other embodiments, the certain time period is n=2, 3, 4, 5, 6, 7, 8, 9, 10, 11, 12, 13, 14, 15, 16, 17, 18, 19, 20, 21, 22, 23, 24, 25, 26, 27, 28, 29 or 30 days, the days of consecutive administration of Compounds A², B² and C within a specified time period is m=1, 2, 3, 4, 5, 6, 7, 8, 9, 10, 11, 12, 13, 14, 15, 16, 17, 18, 19, 20, 21, 22, 23, 24, 25, 26, 27, 28 or 29, the days of administration of one of Compounds A², B² and C is p=0, 1, 2, 3, 4, 5, 6, 7, 8, 9, 10, 11, 12, 13, 14, 15, 16, 17, 18, 19, 20, 21, 22, 23, 24, 25, 26, 27, 28 or 29, and the days of administration of two of Compounds A², B² and C is q=0, 1, 2, 3, 4, 5, 6, 7, 8, 9, 10, 11, 12, 13, 14, 15, 16, 17, 18, 19, 20, 21, 22, 23, 24, 25, 26, 27, 28 or 29, where p or q is at least 1. For example, Compounds A², B² and C can be administered within a specified time period for 1, 2, 3, 4, 5, 6, 7, 8, 9, 10, 11, 12 or 13 consecutive days over a certain time period of 14 days, during which Compound B² is administered for 13, 12, 11, 10, 9, 8, 7, 6, 5, 4, 3, 2, 1 or 0 days, and Compound B² and C are administered for n-m-q days. In this example, n=14, m=1, 2, 3, 4, 5, 6, 7, 8, 9, 10, 11, 12 or 13, and q=13, 12, 11, 10, 9, 8, 7, 6, 5, 4, 3, 2, 1 or 0. The consecutive days during which Compounds A², B² and C are administered within a specified time period can occur any time during the certain time period. Accordingly, in the foregoing example, Compound B² could be administered alone for 4 consecutive days follow by administration of Compounds A², B² and C for 6 consecutive days, followed by administration of Compound B² and C for 4 consecutive days to complete the 14 day certain time period.

It is to be understood that treatment protocols of the present invention include, but are not limited to, administration of Compounds A², B² and C within a specified period in conjunction with administration of any subset of Compounds A², B² and C (e.g., in conjunction with Compound A² alone, in conjunction with Compound B² alone, in conjunction with Compound C alone, in conjunction with Compound A² and Compound B², in conjunction with Compound A² and Compound C, in conjunction with Compound B² and compound C, or in conjunction with any combination thereof).

If the compounds are not administered during a “specified period”, they are administered sequentially.

When the combination of compound A² and Compound B², without Compound C, is administered, the term “sequential administration”, and derivatives thereof, as used herein means that one of Compound A² and Compound B² is administered for one or more consecutive days and the other of Compound A² and Compound B² is subsequently administered for one or more consecutive days. Also, contemplated herein is a drug holiday utilized between the sequential administration of one of Compound A² and Compound B² and the other of Compound A² and Compound B². As used herein, a drug holiday is a period of one or more days after the administration of one of Compound A² and Compound B² and before the sequential administration of the other of Compound A² and Compound B² where neither Compound A² nor Compound B² is administered. The drug holiday can be a various number of days. In some embodiments, the drug holiday is 1, 2, 3, 4, 5, 6, 7, 8, 9, 10, 11, 12, 13 or 14 days.

In some embodiments, one of Compound A² and Compound B² is administered for 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 consecutive days, followed by an optional drug holiday of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days, followed by administration of the other of Compound A² and Compound B² for 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 consecutive days

When the combination of compound A², Compound B², and Compound C is administered, the term “sequential administration”, and derivatives thereof, as used herein means that one or two of Compound A², Compound B² and Compound C is administered for one or more consecutive days and the other two or one of Compound A², Compound B² and Compound C is subsequently administered for one or more consecutive days, such that Compound A², Compound B² and Compound C are each administered at some time during the specified period. Also, contemplated herein is a drug holiday utilized between the sequential administration of one or two of Compound A², Compound B² and Compound C and the other two or one of Compound A², Compound B² and Compound C is subsequently administered for one or more consecutive days, such that Compound A², Compound B² and Compound C are each administered at some time during the specified period. As used herein, a drug holiday is a period of one or more days after the administration of one or two of Compound A², Compound B² and Compound C and before the sequential administration of the other two or one of Compound A², Compound B² and Compound C where neither Compound A², Compound B², nor Compound C is administered. The drug holiday can be a various number of days. In some embodiments, the drug holiday is 1, 2, 3, 4, 5, 6, 7, 8, 9, 10, 11, 12, 13 or 14 days.

In some embodiments, one or two of Compound A², Compound B² and Compound C is administered for 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 consecutive days, followed by an optional drug holiday of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days, followed by administration of the other two or one of Compound A², Compound B² and Compound C for 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 consecutive days.

It is understood that a “specified period” administration and a “sequential” administration can be followed by repeat dosing or can be followed by an alternate dosing protocol, and a drug holiday may precede the repeat dosing or alternate dosing protocol.

It is to be understood that the treatment protocols and regimens described herein can comprise the entire treatment protocol for a given patient or, alternatively, can comprise only a portion of the entire treatment protocol for the patient.

Suitably, the amount of Compound A² administered as part of the combination according to the present invention will be an amount selected from a lower limit of about 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595 or 600 mg to an upper limit of about 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, or 800 mg. It is to be understood that embodiments of the present invention include any number in the ranges listed above. In some embodiments, the selected amount of Compound A² is administered from 1 or 2 times per day.

Suitably, the amount of Compound B² administered as part of the combination according to the present invention will be an amount selected from a lower limit of about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295 or 1300 mg to an upper limit of about 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495 or 1500 mg. In some embodiments, the selected amount of Compound B² is administered 1 or 2 times per day.

Suitably, the amount of Compound C administered as part of the combination according to the present invention will be an amount selected from a lower limit of about 5, 10, 15, 20, 25, 30, 35, 40, 45, 50, 55, 60, 65, 70, 75, 80, 85, 90, 95 or 100 mg/m² to an upper limit of about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200 mg/m². In some embodiments, the selected amount of Compound C is administered every 1, 2, 3, 4, 5, 6, 7, 8, 9, 10, 11, 12, 13, 14, 15, 16, 17, 18, 19, 20, 21, 22, 23, 24, 25, 26, 27 or 28 days.

As used herein, all amounts specified for Compound A², Compound B² and Compound C are indicated as the administered amount of free or unsalted compound per dose.

The method of the present invention may also be employed with other therapeutic methods of ovarian cancer treatment.

While it is possible that, for use in therapy, therapeutically effective amounts of the combinations of the present invention may be administered as the raw chemical, it is preferable to present the combinations as a pharmaceutical composition or compositions. Accordingly, the invention further provides pharmaceutical compositions, which include Compound A² and/or Compound B² and one or more pharmaceutically acceptable carriers. The combinations of the present invention are as described above. The carrier(s) must be acceptable in the sense of being compatible with the other ingredients of the formulation, capable of pharmaceutical formulation, and not deleterious to the recipient thereof. In accordance with another aspect of the invention there is also provided a process for the preparation of a pharmaceutical formulation including admixing Compound A² and/or Compound B² with one or more pharmaceutically acceptable carriers. As indicated above, such elements of the pharmaceutical combination utilized may be presented in separate pharmaceutical compositions or formulated together in one pharmaceutical formulation.

Pharmaceutical formulations may be presented in unit dose forms containing a predetermined amount of active ingredient per unit dose. As is known to those skilled in the art, the amount of active ingredient per dose will depend on the condition being treated, the route of administration and the age, weight and condition of the patient. Preferred unit dosage formulations are those containing a daily dose or sub-dose, or an appropriate fraction thereof, of an active ingredient. Furthermore, such pharmaceutical formulations may be prepared by any of the methods well known in the pharmacy art.

Compound A² and Compound B² may be administered by any appropriate route. Suitable routes include oral, rectal, nasal, topical (including buccal and sublingual), vaginal, and parenteral (including subcutaneous, intramuscular, intravenous, intradermal, intrathecal, and epidural). It will be appreciated that the preferred route may vary with, for example, the condition of the recipient of the combination and the precise nature of the ovarian cancer to be treated. Suitably, Compound C is administered via intravenous or intraperitoneal routes. It will also be appreciated that each of the agents administered may be administered by the same or different routes and that Compound A² and Compound B² may be compounded together in a pharmaceutical composition/formulation. In some embodiments, Compound A², Compound B², and, in some embodiments, Compound C are administered in separate pharmaceutical compositions. In other embodiments, Compound A² and Compound B² are administered in fixed-dose pharmaceutical compositions that include both Compound A² and Compound B² and, in some embodiments, Compound C is administered as a separate pharmaceutical composition.

The compounds or combinations of the current invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier may include a prolonged release material, such as glyceryl monostearate or glyceryl distearate, alone or with a wax. The amount of solid carrier varies widely but, suitably, may be from about 0.05 mg to about 1 g per dosage unit. When a liquid carrier is used, the preparation will suitably be in the form of a syrup, elixir, emulsion, soft gelatin capsule, sterile injectable liquid such as an ampoule, or an aqueous or nonaqueous liquid suspension.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

It should be understood that in addition to the ingredients mentioned above, the formulations may include other agents conventional in the art having regard to the type of formulation in question, for example those suitable for oral administration may include flavoring agents.

As indicated, therapeutically effective amounts of the combinations of the invention (Compound A² in combination with Compound B², or in some embodiments Compound A² and Compound B² in combination with Compound C) are administered to a female human. Typically, the therapeutically effective amount of the administered agents of the present invention will depend upon a number of factors including, for example, the age and weight of the subject, the precise condition requiring treatment, the severity of the condition, the nature of the formulation, and the route of administration. Ultimately, the therapeutically effective amount will be at the discretion of the attending physician.

This invention provides a combination comprising 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof.

This invention also provides for a combination comprising 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof, for use in the treatment of ovarian cancer.

This invention also provides a pharmaceutical composition comprising a combination of 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof.

This invention also provides a combination kit comprising 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof.

This invention also provides for the use of a combination comprising 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof, in the manufacture of a medicament for the treatment of ovarian cancer.

This invention also provides a method of treating ovarian cancer which comprises administering a combination of 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof, to a female subject in need thereof.

This invention provides a combination comprising 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof, and 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate}.

This invention also provides for a combination comprising 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof, and 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate} for use in the treatment of ovarian cancer.

This invention also provides a combination kit comprising 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof, and 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate}.

This invention also provides for the use of a combination comprising 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof, and 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate} in the manufacture of a medicament for the treatment of ovarian cancer.

This invention also provides a method of treating ovarian cancer which comprises administering a combination of 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof, and 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate} to a female subject in need thereof.

The following examples are intended for illustration only and are not intended to limit the scope of the invention in any way.

### EXPERIMENTAL DETAILS

### Materials and Methods:

### Drugs and Reagents:

Pazopanib monohydrochloride, (5-[[4-[(2,3-Dimethyl-2H-indazol-6-yl)methylamino]-2-pyrimidinyl]amino]-2-methylbenzolsulfonamide is available from GlaxoSmithkline. Pazopanib monohydrochloride and 2-[(5-chloro-2-[[3-methyl-1-(1-methylethyl)-1H-pyrazol-5-yl]amino]-4-pyridinyl)amino]-N-methoxybenzamide [“the FAK inhibitor”] were provided by GlaxoSmithKline. Docetaxel, 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate} was obtained from Sanofi-Aventis, Bridgewater, N.J.

### Cell Lines:

Cell lines HeyA8 and SKOV3-IP1 (human ovarian cancer cell lines) were obtained from the MD Anderson Cancer Center Characterized Cell Line Core, Houston, Tex.

### In-Vitro Cytotoxicity:

4000 HeyA8 cells and 4000 SKOV3-IP1 cells were seeded in 96-well plates and incubated for 24 h in complete media followed by 24 h in serum-free media, after which they were treated with FAK inhibitor for 24, 48, and 72 h and progressive doses. Cell viability was determined by MTT (3-(4,5-dimethylthiazol-2-yl)-2,5-diphenyltetrazolium bromide) assay. 15% MTT solution was added to each well 2 h prior to analysis. At time of analysis, the solution was removed, dimethyl sulfoxide was added, and colorimetric detection was performed using the BioTek Instruments μQuant at a primary wavelength of 570 nm.

### Tumor Xenograft Models:

Tumor cells were injected intraperitoneally into nude mice. For the first study, mice were inoculated with 1,000,000 SKOV3-Ip1 cells. For the second study, mice were inoculated with 250,000 HeyA8 cells. At the time of inoculation, mice were randomized into groups to be treated by oral gavage and intraperitoneal injection. The animals were grouped as described below. The criteria for end point was animals in any group being moribund. At the time of dissection, animals were sacrificed by cervical dislocation and necropsy immediately performed. Tumor nodules were counted and aggregate tumor weight (g) was recorded. Tumors were fixed in 10% formalin, and fresh tumor was flash-frozen for future analysis.

### Combination Study

The goal of this study was to determine the in vivo effects of each of pazopanib, the FAK inhibitor, and docetaxel, alone and in combinations. Nude mice were inoculated with tumor as described above and randomized to the groups below. The following doses of each inhibitor were used: FAK-inhibitor, 75 mg/kg, oral, daily; pazopanib, 100 mg/kg, oral, daily; docetaxel, 35 ug, IP, weekly. Animals were grouped in the following manner:
Control gavage
FAK inhibitor only
FAK inhibitor, Docetaxel
FAK inhibitor, Pazopanib
FAK inhibitor, Pazopanib, Docetaxel
Pazopanib, Docetaxel
Docetaxel only
Pazopanib only
   The criteria for end point was animals showing signs of morbidity. At the time of dissection, animals were sacrificed by cervical dislocation and necropsy immediately performed. Tumor nodules were counted and aggregate tumor weight (g) was recorded. Tumors were fixed in 10% formalin, and fresh tumor was flash-frozen for future analysis.

### Statistical Analysis

In vitro dose-response, in vivo tumor growth and scoring of the immunohistochemistry are presented as mean±Standard Error. Statistical significance was assessed by student's T-test and P≦0.05 was considered to be significant. Statistics were performed with Microsoft Excel 2007 (Microsoft Corporation, Redmond, Wash.).

### Results:

### Drug-Induced In Vitro Cytotoxities

The FAK-inhibitor resulted in reduced levels of FAK phosphorylation at Y397 (pFAK^{Y397}) at 1 μM concentration in SKOV3-IP1 cells and at 10 μM in HeyA8 cells. The FAK-inhibitor resulted in a 12.5% reduction in invasion (p<0.001) and a 54% reduction in migration (p<0.001) in SKOV3-IP1 cells.

### Combination Study

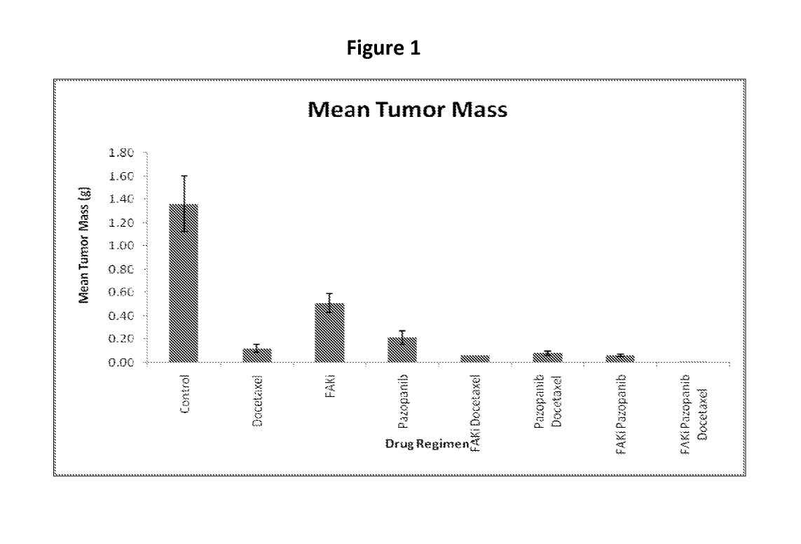

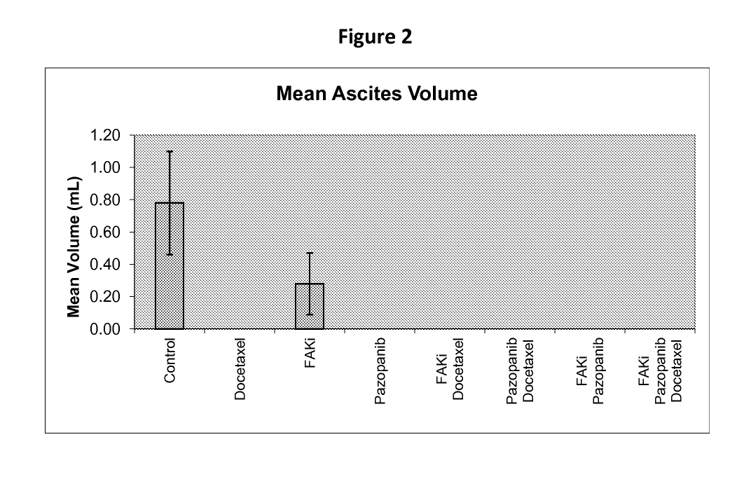

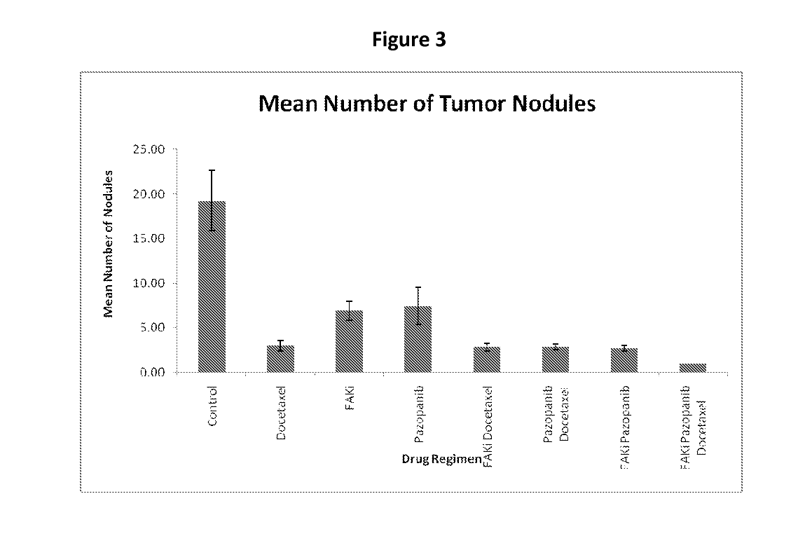

At time of animal sacrifice, necropsy was immediately performed. Tumor nodules were counted, and aggregate tumor weight was measured. Ascites volume was measured directly at time of entry into the abdominal cavity. Monotherapy with the FAK-inhibitor resulted in a 58% decrease in mean tumor weight compared to control (p=0.038). The combination of the FAK-inhibitor with pazopanib resulted in a 71% decrease in mean tumor weight compared to pazopanib monotherapy (p=0.04). The combination of the FAK-inhibitor with docetaxel resulted in a 44% decrease in mean tumor weight compared to docetaxel monotherapy (p=0.17). The triple combination of the FAK-inhibitor with pazopanib and docetaxel resulted in the greatest overall decrease mean tumor mass, 99% compared to control and 92% compared to the doublet alone (p=0.001). (FIG. 1) Similar trends were noted with ascites volume. (FIG. 2) and mean number of tumor nodules (FIG. 3). Treatment with pazopanib decreased MVD by 49% (p<0.01), which was further enhanced in combination with the FAK-inhibitor (p<0.01).

While the preferred embodiments of the invention are illustrated by the above, it is to be understood that the invention is not limited to the precise instructions herein disclosed and that the right to all modifications coming within the scope of the following claims is reserved.

We claim:

1. A combination comprising:
(i) a compound of Structure (I): 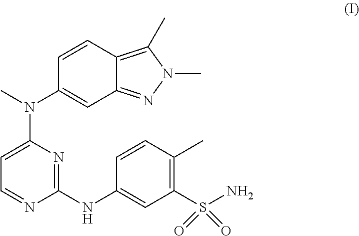 [SMILES: Cc1ccc(Nc2nccc(N(C)c3ccc4c(C)n(C)nc4c3)n2)cc1S(N)(=O)=O]
or a pharmaceutically acceptable salt thereof; and
(ii) a compound of Structure (II): 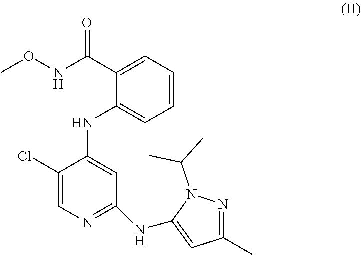
or a pharmaceutically acceptable salt thereof;
where the compound of Structure (I) and the compound of Structure (II) are in synergistic amounts.

2. A combination according to claim 1 where the compound of Structure (I) is in the form of a monohydrochloride salt.

3. A combination according to claim 1 further comprising a compound of Structure (III):

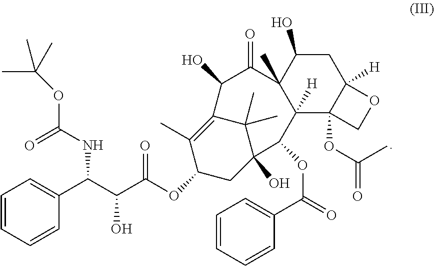

4. A combination kit comprising a combination according to claim 1 together with a pharmaceutically acceptable carrier or carriers.

5. A combination according to claim 1 where the amount of the compound of Structure (I) is an amount from 100 mg to 800 mg, and that amount is administered once per day in one or more tablets, and the amount of the compound of Structure (II) is an amount from 100 mg to 800 mg, and that amount is administered once per day.

6. A combination according to claim 3 where the amount of the compound of Structure (Ill) is in an amount of from 5 mg/m² to 200 mg/m², and that amount is administered once per week.

7. A method of treating ovarian cancer in a female human in need thereof, comprising the in vivo administration of a synergistic amount of a combination of 5-[[4-[(2,3-dimethyl-2H-indazol-6-yl)methylamino]-2-pyrimidinyl]amino]-2-methylbenzenesulfonamide, or a pharmaceutically acceptable salt thereof, and 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof, to such human, wherein the combination is administered within a specified period, and wherein the combination is administered for a duration of time.

8. A method according to claim 7 wherein the amount of 5-[[4-[(2,3-dimethyl-2H-indazol-6-yl)methylamino]-2-pyrimidinyl]amino]-2-methylbenzenesulfonamide, or a pharmaceutically acceptable salt thereof, is from about 100 mg to about 800 mg, and that amount is administered once per day.

9. A method according to claim 7 wherein the amount of 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof, is from about 100 mg to about 800 mg, and that amount is administered once per day.

10. A method according to claim 7 wherein the combination further comprises 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate}.

11. A method according to claim 10, wherein the amount of 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate} is from 5 mg/m² to 200 mg/m², and that amount is administered once per week.

12. A method according to claim 7 wherein the specified period is within about 1 to about 12 hours.

13. A method according to claim 7 wherein the duration of time is for from 1 to 30 consecutive days.

14. A method according to claim 7 wherein 5-[[4-[(2,3-dimethyl-2H-indazol-6-yl)methylamino]-2-pyrimidinyl]amino]-2-methylbenzenesulfonamide is in the form of a monohydrochloride salt.

15. A method treating ovarian cancer in a female human in need thereof, comprising the in vivo administration of a synergistic amount of a combination of 5-[[4-[(2,3-dimethyl-2H-indazol-6-yl)methylamino]-2-pyrimidinyl]amino]-2-methylbenzenesulfonamide, or a pharmaceutically acceptable salt thereof, and 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof, to such human, wherein the compounds of the combination are administered sequentially.

16. A method according to claim 15, wherein the amount of 5-[[4-[(2,3-dimethyl-2H-indazol-6-yl)methylamino]-2-pyrimidinyl]amino]-2-methylbenzenesulfonamide, or a pharmaceutically acceptable salt thereof, is from about 100 mg to about 800 mg, and that amount is administered once per day.

17. A method according to claim 15 wherein the amount of 2-[(5-chloro-2-[[3-methyl-1-(1-methylethyl)-1H-pyrazol-5-yl]amino]-4-pyridinyl)amino]-N-methoxybenzamide, or a pharmaceutically acceptable salt thereof, is from about 100 mg to about 800 mg, and that amount is administered once per day.

18. A method according to claim 15 wherein the combination further comprises 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate}.

19. A method of treating ovarian cancer which comprises administering a combination according to claim 1.
